(19) United States
(12) Patent Application Publication
(10) Pub. No.: US 2004/0053406 A1
(43) Pub. Date: Mar. 18, 2004

(54) Pluripotent embryonic stem (es) cell lines, improved methods for their production, and their use for germ line transmission and for the generation of genetically modified animals

(76) Inventors: Luc Schoonjans, Wilsele (BE); Randall Moreadith, Raleigh, NC (US)

Correspondence Address: Barbara E Johson, 700 Koppers Building, 436 Seventh Avenue, Pittsburgh, PA, 15219-1818, US

(21) Appl. No.: 10/312,202

(22) PCT Filed: Jun. 28, 2001
(86) PCT No.: PCT/EP01/07396

(30) Foreign Application Priority Data
Jun. 28, 2000 (EP) 00202254.9

(51) Int. Cl. C12N 5/08; C07K 5/06

(52) U.S. Cl. 435/366; 435/325

(57) ABSTRACT

The invention relates to a novel composition for maintaining and growing pluripotent and germ line competent mouse embryonic stem cells. The composition includes high glucose DMEM, non essential amino acids, glutamine, beta-mercaptoethanol and fetal bovine serum or the equivalents thereof, which is conditioned by an immortalized rabbit fibroblast cell line transduced with genomic rabbit Leukemia Inhibitory Factor (LIF). The invention further relates to the use of the composition for producing embryonic stem cell lines and to the use of these cell lines in the production of transgenic animals.

### FIELD OF THE INVENTION

The present invention relates to a novel composition for maintaining and growing pluripotent and germ line competent mouse embryonic stem cells. The composition includes high glucose DMEM, non-essential amino acids, glutamine, beta-mercaptoethanol and fetal bovine serum or the equivalents thereof, which is conditioned by an immortalized rabbit fibroblast cell line transduced with genomic rabbit Leukemia Inhibitory Factor (LIF). The invention further relates to the use of the composition for producing embryonic stem cell lines and their use for germ line transmission and for the generation of genetically modified non-human animals.

### BACKGROUND OF THE INVENTION

Embryonic stem (ES) cell lines, isolated from the inner cell mass (ICM) of blastocyst-stage embryos, can be maintained and passaged through multiple generations in culture without loss of their pluripotency. They maintain a normal karyotype and when reintroduced into a host blastocyst can colonize the germ line (Bradley A. Production and analysis of chimeric mice. In: Teratocarcinomas and Embryonic Stem Cells: A practical approach (Ed. E J Robertson) JRI press Ltd., Oxford 1987, p 113-51). To date, germ line transmission, i.e. the transmission of the ES genome to the next generation, has however only been achieved with ES cells of certain mouse strains, primarily the 129 and C57BL/6 strains, whereas ES cell lines are at best obtained in 10 to 30% of explanted blastocysts (Robertson E J. Embryo-derived stem cell lines. In Teratocarcinomas and Embryonic Stem Cells: A Practical Approach (Ed. E J Robertson) 1987. IRL Press, Oxford, pp 71-112; Nagy A, Rossant J, Nagy R, Abramov-Newerly W, Roder J C. Derivation of completely cell culture derived mice from early-passage embryonic stem cells. Proc Natl Acad Sci USA 1993; 90: 8424-8).

Murine ES cells were first isolated in 1981 (Evans M J, Kaufman M H. Establishment in culture of pluripotential cells from mouse embryos. Nature 1981; 292: 154-6; Martin G R. Isolation of a pluripotent cell line from early mouse embryos cultured in medium conditioned with teratocarcinoma stem cells. Proc Natl Acad Sci USA 1981; 78: 7634-8) and are now widely used for the introduction of targeted mutations into the mouse genome (Pascoe W S, Kemler R, Wood S A. Genes and functions: trapping and targeting in embryonic stem cells. Biochim Biophys Acta 1992; 1114: 209-21). ES cell lines can be transformed in vitro with DNA and selected for recombination (homologous or non-homologous) of exogenous DNA into chromosomal DNA, allowing stable incorporation of the desired gene. Since the genetic background may be important in some phenotypes, ES cell lines from other inbred and mutant mouse strains are desirable.

It is known that chimeric animals may be generated by injection of about 10-15 isolated ES cells into the blastocoel of a host blastocyst, allowing the cells to mix with the cells of the inner cell mass (Bradley 1987, supra). Alternatively, diploid aggregation, using very early (8-16 cell) stage embryos (Tokunaga T, Tsunoda Y. Efficacious production of viable germ-line chimeras between embryonic stem (ES) cells and 8-stage embryos. Dev Growth & Differ 1992; 34: 561-6), and tetraploid aggregation, using electrofusion derived tetraploid 4-celled embryos (Nagy A, Gocza E, Diaz E M, Prideaux V R, Ivanyi E, Markkula M, Rossant J. Embryonic stem cells alone are able to support fetal development in he mouse. Development 1990; 110: 815-21), can be used to “sandwich” ES cells between early stage embryos devoid of their zona pellucida. The resultant chimeric blastocysts or aggregates are then transferred to recipients for rearing. ES cell technology is still under development and there are no reports on germ line transmission in any other species than mouse.

The pluripotency of ES cells is often reduced after several passages, whereas completely ES cell-derived foetuses have a markedly reduced survival after birth. Aggregation of R1 ES cell lines derived from early passages with tetraploid embryos derived by electrofusion yields mice, which are entirely derived from ES cells (Nagy et al., 1993, supra). However, no animal derived from R1 ES cells obtained from later than 14 passages survived to adulthood and less than 5% of transferred aggregates from early passage ES cells survived after caesarean section at term. Thus, the routine production of mice entirely derived from genetically modified inbred ES cells did not seem to be possible.

An alternative route towards reinstating the ES genome in the germ line is by means of nuclear transfer, as first demonstrated by Campbell et al. (Sheep cloned by nuclear transfer from a cultured cell line. Nature 1996; 380: 64-6) who generated viable sheep zygotes by fusing individual inner cell mass cells with enucleated oocytes. When applied to ES cells, this route will ensure that all the cells in the offspring, including the germ cells, are of the ES cell genotype. Nuclear transfer is achieved by electrofusing a karyoplast with a surgically enucleated oocyte (cytoplast) derived from in vivo or in vitro sources (Loi P, Boyouzoglu S, Fulka J Jr, Naitana S, Cappai P. Embryo cloning by nuclear transfer: experiences in sheep. Livestock Production Science 1999; 60: 281-94), but the overall success of this process is below 10%.

It is therefore the object of the present invention to improve upon these known methods.

According to the invention markedly improved methods were found for the derivation and culturing of ES cells from any one of over 10 different genetic backgrounds (including several inbred strains), with superior potential for germ line transmission.

### SUMMARY OF THE INVENTION

The present invention is directed to a novel composition for maintaining and growing pluripotent and germ-line competent mammalian embryonic stem cells. The composition consists of a basal cell medium, which comprises high glucose DMEM, non-essential amino acids, glutamine, beta-mercaptoethanol and fetal bovine serum or equivalents thereof, which basal cell medium is conditioned by a rabbit fibroblast cell clone (Rab #9) transfected with genomic rabbit Leukemia Inhibitory Factor (LIF). In addition, penicillin/streptomycin may be and insulin is included in the composition.

### DETAILED DESCRIPTION OF THE INVENTION

The present invention relates to the use of the improved ES cell medium of the invention in markedly improved methods for the derivation and culturing of ES cells as exemplified in mouse strains. These improved culture conditions have already generated stable murine ES cells from any one of more than 10 different genetic backgrounds tested, with superior potential for germ line transmission. This technology is also applicable to other species (rabbits, pigs, cattle etc.) and does form the basis for targeted transgenesis with gain-of-function or loss-of-function in non-murine species, and does allow targeted genetic manipulation of live stock.

The invention thus relates to a composition for maintenance and growth of pluripotent and germ line-competent mammalian embryonic stem (ES) cell lines, which composition consists of a basal cell medium, which comprises high glucose DMEM, non-essential amino acids, glutamine, beta-mercaptoethanol and fetal bovine serum or equivalents thereof, which basal cell medium is conditioned by a fibroblast cell clone that produces Leukemia Inhibitory Factor (LIF).

The basal cell medium comprises the following compounds in amounts sufficient to maintain ES cells for prolonged periods in culture:

1) DMEM high glucose;

2) penicillin/streptomycin;

3) non essential amino acids;

4) glutamine;

5) beta-mercaptoethanol; and

6) foetal bovine serum.

The LIF producing fibroblasts are preferably immortalized rabbit fibroblasts. In particular they are immortalized fibroblasts that have been transfected, transformed or infected by a vector overexpressing a LIF gene, preferably a rabbit LIF gene.

In a preferred embodiment of the invention the fibroblast cell line used for conditioning is the Rab9 #19 cell line, which has been deposited with the Belgian Coordinated Collection of Micro-organisms, under accession number LMBP 5479 CB.

The composition of the invention may have a varying amount of constituents provided that their amount is sufficient to maintain ES cells for prolonged periods in culture. A preferred example of a composition of the invention comprises per liter conditiond medium of the LIF producing cell line, added volume of 50 to 120, preferably 80 ml of foetal bovine serum, 10 to 25, preferably 17 ml non-essential amino acids, 2 to 8, preferably 5μl β-mercaptoethanol, 0.5 to 2.5, preferably 1.25 ml insulin, 80 to 130 ml basal ES cell medium (in ratios to adjust the LIF to a final concentration of 14 to 15 ng/ml).

Preferably, the basal ES cell medium consists of 400 to 600, preferably 500 ml DMEM high glucose, 0 to 15, preferably 13 ml penicillin/streptomycin, 10 to 15, preferably 13 ml non essential amino acids, 10 to 15, preferably 13 ml glutamine, 5 to 10, preferably 6.3 μl β-mercaptoethanol, 50 to 100, preferably 70 ml foetal bovine serum, neutral pH of preferably 7.4.

The composition of the invention may be used in the production of pluripotent embryonic stem (ES) cell lines.

The invention further relates to a process of culturing mammalian ES stem cells to obtain pluripotent and germ line-competent ES cells, wherein the culturing of the mammalian ES stem cells is at least partially performed in a composition according to the invention and described above.

Such a process comprises the steps of:

a) culturing cells of blastocyst stage embryos;

b) culturing isolated inner mass cells; and

c) passaging the inner mass cells periodically in a composition of the invention.

Preferably, the inner mass cells are periodically passaged for at least 8 times. The process may further comprise the step of producing transgenic animals.

According to a further aspect thereof the invention relates to embryonic stem (ES) cell lines with germ line transmission capability. Preferably the germ line transmission capability is retained after 11 or more passages.

Cell lines of the invention are obtainable by the process of the invention as described above. The cell line is preferably a murine cell line, but other animal cell lines are also possible. In case of a murine cell line, the cell line has been derived from cells or tissues with 129/SvEv, C57BL/6N, C57BL/6J-HPRT, BALB/c, CBA/CaOla, 129/SvJ, DBA/2n, DBA/1 Ola, C3H/HeN, C57BL/6JOla, FVB or Swiss Webster genetic backgrounds. The murine cell lines preferably have a germ line transmission capability after 11 or more passages.

The cell line of the invention may be cultured in a composition of the invention supplemented with cytokines and growth factors.

The embryonic stem (ES) cell lines of the invention are characterized by three dimensional colony formation, positive staining for alkaline phosphatase and negative staining for cytokeratin 18 and vimentin after more than 10 passages. These embryonic stem (ES) cell lines may be used in the generation of chimeric or ES cell derived animals, in the gene alteration by homologous or non-homologous recombination, in the generation of animals with gene alteration via germ line transmission, for the generation of chimeric animals, for the generation of chimeric animals following blastocyst injection into recipient blastocysts or embryo aggregation or nuclear transfer, for the study or isolation of (novel) genes or for the expression or overexpression of genes.

The invention will be illustrated in the following examples, that are not intended to limit the scope of the invention. Based on the present invention, several variations and improvements will be obvious to those skilled in the art.

### EXAMPLES

### Example 1

Production of Improved ES Cell Medium which Maintains Embryonic Stem (ES) Cells Undifferentiated

Phage plagues representing a “Sau 3A-partial” rabbit genomic library were grown at a density of 300.000 plaques per 24×24 cm dish and transferred to nitrocellulose in duplicate. This rabbit genomic lamba DASH II library (Stratagene, #955950) was screened with a 1200 bp probe containing the 580 bp murine LIF cDNA probe. After hybridization overnight at 42° C., the membrane was washed twice at room temperature for 20 min with 0.5×SSC and 0.5% SDS and for 45 min at 55° C. and for 30 min at 59° C. with 0.2×SSC and 0.5% SDS and then autoradiographed. Plaques positive on duplicate filters were rescreened at lower density.

One clone was subjected to sequence analysis (Sanger) and identified as encoding the rabbit LIF protein. A 2.9 kb BamHI fragment containing the complete rabbit LIF genomic DNA was then inserted into an expression cassette with the PGK promoter and the bovine poly A sequence.

Permanent expression of the rabbit LIF gene was achieved in immortalized rabbit fibroblast cells (Rab9 fibroblasts, purchased from ATCC, Manassas, Va., USA) by cotransfection of the LIF expression cassette with a cassette encoding for neomycin resistance. The cotransfection was realized with 10 consecutive pulses (99 μsec, 2.5 kV/cm, direct current, BTX electro cell manipulator ECM 200, San Diego, Calif., USA), 5 μg of BglII & XhoI fragment (4.4 kb) from the neomycine resistance cassette and 15 μg of a HindIII/NotI fragment comprising the LIF expression cassette. Dulbecco's PBS was used as electroporation buffer.

The neomycin resistance cassette comprised the PGK promoter (0.5 kb)+n-galineo (3.6 kb)+bovine poly A (325 bp) in the pSP72 vector (2.4 kb). N-galineo was designed and constructed as a fusion between the nuclear localizing form of β-galactosidase in frame with neomycin.

The rabbit LIF expression cassette comprised the PGK promoter (0.5 kb)+a BamHI fragment of 2.9 kb containing the rabbit LIF genomic DNA+bovine poly A (325 bp) in the pSP72 vector (2.4 kb).

Basic ES cell medium consists for example of 500 ml DMEM high glucose (cat no. 12430-054), 13 ml penicillin/streptomycin, 13 ml non essential amino acids, 13 ml glutamine, 6.3 μl β-mercaptoethanol, 70 ml foetal bovine serum, pH 7.4.

Non-transfected rabbit fibroblast cells did not produce measurable quantities of rabbit LIF (i.e., less than 20 pg/ml/24 hours, when grown on 15 cm dishes with basic ES medium at 39° C. in a humidified atmosphere of 5% CO₂ in air). After transfection, several G418 (200 μg/ml) resistant colonies were isolated, which also produced rabbit LIF (i.e., more than 20 pg/ml/24 hours or up to 30 ng rabbit LIF/ml/24 hours in the medium when grown on 15 cm dishes with 25 ml basic ES medium at 39° C. in a humidified atmosphere of 5% CO in air).

A transfected fibroblast clone (Rab9 #19) was deposited with the Belgian Coordinated Collection of Micro-organisms, under accession number LMBP 5479CB.

Basal ES cell medium, conditioned by the Rab9 #19 fibroblast cells, is collected for 4 consecutive days. Each day the dishes are refreshed with 25 ml of basic ES medium. After 4 days each 15 cm dish is split at a ratio of 1 to 7. The first day after splitting the medium is not collected, but discarded.

Improved ES cell medium of the invention may for example consist of 450 ml of conditioned basal cell medium (from the mixture of the 4 collection days), 60 ml of basal cell medium, 10 ml non essential amino acids, 10 ml glutamine, 2.3 μl β-mercaptoethanol, 70 ml foetal calf serum, 0.6 ml bovine insulin, pH 7.4.

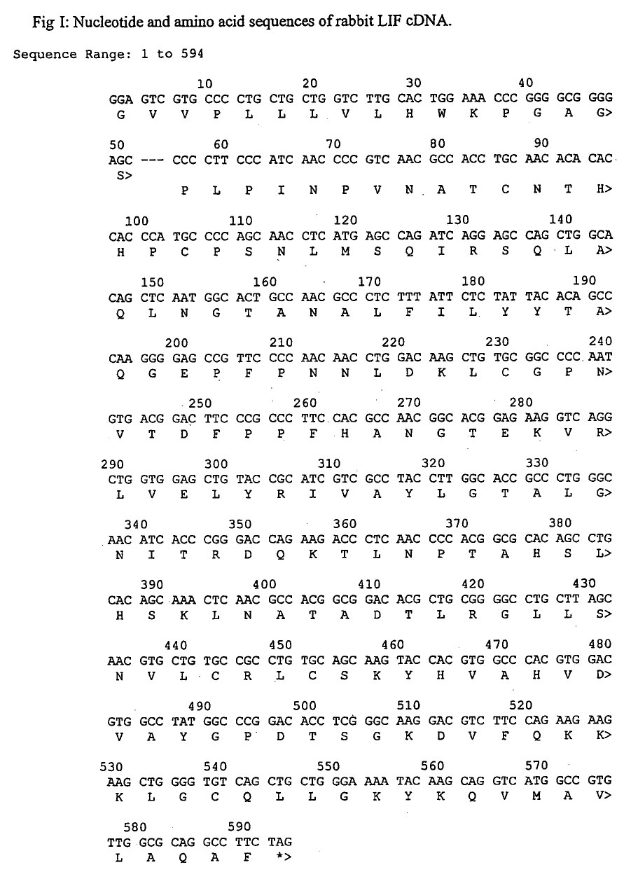

The nucleotide sequence and amino acid sequence of the rabbit LIF cDNA which has not previously been reported, is shown in FIG. 1. The nucleotide sequence was determined as described in Sanger et al. (Sanger F, Nicklen S, Coulsor A. DNA sequencing with chain-terminating inhibitors. Proc Natl Acad Sci USA 1977; 74: 5463-7), on a cloned Sau IIIA genomic DNA fragment.

### Example 2

Technological Aspects of Mouse Embryonic Stem Cell Derivation, Culture and Generation of Chimeric and ES Cell Derived Animals

1. Mouse Strains and ES Cells

ES cells were derived from the following commercially available mouse strains: 129/SvEvTaconic (Taconic, Germantown, N.Y., USA); C57BL/6NTacfBr (Taconic); BALB/cAnNTacfBr (Taconic); DBA/2NTacfBR (Taconic); C3H/HeNTac-MTVfBe (Taconic), FVB/NTacfBR (Taconic); Tac:(SW)fBR, Swiss Webster (Taconic); 129/SvJ (The Jackson laboratory, Bar Harbor, Me., USA); C57BL/6J-HPRT <B-M3> (The Jackson Laboratory); C57BL/6JOlaHsd (Harlan, Indianapolis, Ind., USA); CBA/CaOlaHsd (Harlan); DBA/lOlaHsd (Harlan).

2. Derivation of Murine ES Cells

ES cells were derived from 3.5-4.5 days old blastocyst stage murine embryos, which were collected and plated individually on a 96 well dish covered with a mitotically arrested mouse embryonic fibroblast feeder monolayer. The blastocysts were allowed to attach to the monolayer, and refed every day with Improved ES Cell Medium of the invention (see Example 1).

After 5-6 days in culture, the inner cell mass (ICM) outgrowth was selectively removed from the (remaining) trophectoderm and replated after trypsinization with trypsin-EDTA on a 96 well dish with mitomycin arrested murine fibroblasts. Subsequently the ES cells were gradually plated on larger culture dishes. ES cells proved to remain undifferentiated for more than 20 passages by using Improved ES cell medium of the invention.

Fibroblast feeder layers were obtained from murine embryos of 12.5 days post-coitus pregnant mice. The mice were sacrificed, and the uteri collected and placed in a petri dish containing phosphate buffered saline (PBS). The embryos were dissected out of the uterus and all membranes removed. The embryos were transferred into a new dish containing PBS, the head and all internal organs removed and the carcasses washed in PBS to remove blood. The carcasses were then minced using 2 insulin syringes into cubes of 2 to 3 mm in diameter, and incubated in Trypsin-EDTA/MEM solution (10/90 V/V) at 4° C. for 2 hrs. The suspension was then incubated at 37° C. for 15 min, a single cell suspension made using a 5 ml pipette, and plated at 5×10⁶ cells per 180 mm petri dish in 25 ml Feeder Medium.

Feeder Medium consisted of 500 ml Dulbecco's Minimal Essential Medium (DMEM), 10% fetal calf serum (FCS), 13 ml Penicillin/Streptomycin, 13 ml Glutamine, 13 ml Non Essential amino Acids, 2.3 μl β-mercaptoethanol. The medium was changed after 24 hr to remove debris. After 2 to 3 days of culture the fibroblasts reached a confluent monolayer. The plates were then trypsinized, replated on 2 petri dishes, and, when confluent, the cells of each plate were frozen in 2 vials, kept at −80° C. overnight and transferred to liquid nitrogen the next day.

3. Culture of ES Cells

ES cells were grown to subconfluency on mouse embryonic fibroblasts mitotically arrested with mitomycin. Culture dishes were kept at 39° C. in a humidified atmosphere of 5% CO₂ in air. The ES cells were passaged every 3-4 days onto freshly prepared feeder dishes. The ES cells were fed every day with the Improved ES cell medium.

4. Blastocyst Injection of ES Cell Clones

The ability of the ES cells to colonize the germ line of a host embryo was tested by injection of these ES cells into host blastocysts, or by their aggregation with morula-stage diploid embryos or 4-celled tetraploid embryos, and implanting these chimeric preimplantation embryos into pseudopregnant foster recipients according to standard procedures. The resulting chimeric offspring were test bred for-germ line transmission of the ES cell genome.

ES cells of mouse strains with a coat colour (C57Bl/6J-HPRT #2, DBA/2N #8, DBA/1 Ola # 36) were injected into host blastocysts of albino Swiss Webster mice. ES cells of mouse strains with a white or creamish coat color (Swiss Webster #43, Swiss Webster #44, 129/SvJ #3, 129/SvJ #4, 129/SvJ #7, BALB/c #17, BALB/c #29, and FVB #17) were injected into host blastocysts of black C57BL/6N mice. This allows easy identification of ES cell contribution. All ES lines tested resulted in chimeric offspring with germ line capability (see below).

5. Diploid Aggregation of ES Cell Clones

The diploid aggregation method was executed as follows. Swiss Webster (albino coat colour) females were superovulated with pregnant mare serum gonadotropin followed 44-48 hrs later by 5 units human chorionic gonadotropin. The oviducts of superovulated and mated Swiss Webster mice were flushed 2.5 days after copulation to collect late 8-cell stage diploid embryos. All ES cell lines tested were derived from mice strains with a coat colour, facilitating identification of chimeric offspring.

Zonae pellucidae of these 8-cell stage diploid embryos were removed by treatment with acid Tyrode's buffer. The zona-free embryos were washed and placed in M16 medium. Aggregation was performed between one 8-cell stage diploid embryo and a clump of ES cells. The aggregates were cultured in micro drops of M16 until the blastocyst stage before they were reimplanted into the uterus horns of 2.5-day pseudopregnant Swiss Webster females.

Chimeric pups were identified by the presence of a dark (=non albino) colour, which originated from an ES cell contribution. The percentage of chimerism (portion of the newborn pup, originating from the ES cells) was visually identified by judging the percentage of dark coat (originating from the ES cells) compared to the white coat (originating from the albino Swiss Webster embryo).

6. Tetraploid Aggregation of ES Cell Clones

Completely ES cell derived embryos were generated via aggregation of the ES cells with tetraploid host embryos. 2-celled embryos were electrically fused, and subsequently aggregated as 4-celled tetraploid embryos with the ES cells to form chimeric embryos, which were then implanted in pseudopregnant recipients. The ES cells (almost) exclusively contributed to the development of the embryo proper, and the tetraploid cells to that of the extra embryonic membranes.

In order to distinguish between the ES and tetraploid cells, host embryos (used for aggregation) were derived from the ROSA26 strain, which expresses LacZ ubiquitously and throughout the entire development and adulthood. The oviducts of superovulated and mated ROSA26 mice were flushed 36 hrs after treatment with human chorionic gonadotropin to collect late two-cell stage embryos.

Electro fusion was carried out to produce tetraploid embryos. The 2-cell stage embryos were placed between two platinum electrodes laid 250 μm apart in 0.2 M mannitol medium in the electrode chamber (Nagy et al., 1993, supra). The two blastomeres were fused by a short electrical pulse (100V for 100μsec in 0.3 M mannitol) applied by a pulse-generator (CF; manufactured by Biochemical Laboratory service, Budapest, Hungary). The fused tetraploid embryos were cultured overnight in M16 micro drops under mineral oil in 37° C. in 95% air/5% CO₂. Twenty-four hours after fusion, most of the tetraploid embryos developed to the four-cell stage. Only these four-cell-stage embryos were used for aggregation.

Zonae pellucidae of these embryos were removed by treatment with acid Tyrode's buffer. ES cell (plated at low density on bare gelatinized dishes without feeder layer 2 days prior to aggregation) were briefly trypsinized to form clumps of loosely connected cells. Clumps of 10-15 ES cells were sandwiched between two tetraploid embryos in aggregation wells. The aggregates were cultured in micro drops of M16 until the blastocyst stage before they were reimplanted into the uterus horns of 2.5-day pseudopregnant Swiss Webster females.

The germ line transmission capacity of our newly derived ES cells were determined at a passage number of 10 or higher.

### Example 3

Derivation of Mouse ES Cell Lines and Generation of es Cell Derived Animals

1. ES Cell Derivation

Most of the germ line-competent murine ES cell lines that are in current use have been obtained in the 129 strain. To establish whether the genetic background is important, ES cell lines were established from various inbred and mutant mice strains.

ES cells have been derived from 11 different inbred mouse strains and 1 outbred strain (as summarized in Table 1). The efficiency of ES cell line derivation ranged between 5 and 66 percent.

In the 129 strains 61% (129/SvEv) and 58% (129/SvJ) of the explanted blastocysts gave rise to an ES cell line. In the C57BL/6 backgrounds the efficiency of ES cell derivation was above 30%. ES cells with germ line transmission capability were obtained from CBA/CaOla mice, a strain previously believed to be non-permissive to ES cell derivation—(McWhir J, Schnieke A E, Ansell R, Wallace H, Colman, Scott A R, Kind A J. Nature Genetics 1996; 14: 223-6).

Two out of 37 BALE/c blastocysts give rise to an ES cell line and both lines transmitted the ES genome through the germ line (see below). A success rate of 11% was obtained in the DBA/1Ola strain. Roach et al. (Roach M L, Stock J L, Byrum R, Koller B H, McNeish. A new embryonic stem cell line from DBA/1 lac J mice allows genetic modification of a murine model of human inflammation. Exp. Cell Res. 1995; 221: 520-5) reported in 1991 a success rate of only 0.01% in the DBA/1lacJ strain.

ES cells were obtained from the DBA/2N, the FVB/N and Swiss Webster strains with efficiencies of 37%, 22% and 7%, respectively. Successful ES cell derivation from these strains has not previously been reported.

Improved ES cell medium allowed derivation of ES cells of genetically manipulated mouse strains (Huang P I, Huang Z H, Mashimo H, Bloch K D, Moskowitz M A, Bevan J A, Fishman M C. Hypertension in mice lacking the gene for endothelial nitric oxide synthase. Nature 1995; 377: 239-42; Piedrahita J A, Zhang S H, Hagaman J R, Oliver P M, Maeda N. Generation of mice carrying a mutant apolipoprotein E gene inactivated by gene targeting in embryonic stem cells. Proc Natl Acad Sci USA 1992; 89: 4471-5; Conway E M, Pollefeyt S, Cornelissen J, De Baere I, Steiner-Mosonyi M, Ong K, Baens M, Collen D, Schuh A C. Three differentially expressed survivin cDNA variants encode proteins with distinct antiapoptotic functions. Blood 2000; 95: 1435-42; Carmeliet P, Dor Y, Herbert J M, Fukumura D, Brusselmans K, Dewerchin M, Neeman M, Bono F, Abramovitch R, Maxwell P, Koch C J, Ratcliffe P, Moons L, Jain R K, Collen D, Keshet E. Role of HIF-1 alpha in hypoxia-mediated apoptosis, cell proliferation and tumour angiogenesis. Nature 1998; 394: 485-90; and Carmeliet P, Mackman N, Moons L, Luther T, Gressens P, Van Vlaenderen I, Demunck H, Kasper M, Breier G, Evrard Ph, Muller M, Risau W, Edgington T, Collen D. Role of tissue factor in embryonic blood vessel development. Nature 1996; 383: 73-5) with high efficiency (Table 2). With the exception of the ApoE−/− C57BL/6 mice (11%), the efficiency of ES cell derivation was consistently above 30%, varying between 35 and 60%.

2. Germ Line Transmission After Blastocyst Injection

All ES lines tested resulted in chimeric offspring after blastocyst injection and showed the capability to pass the ES cell genome to the next generation (Table 3).

Blastocyst injection with ES cells from three of the genetically manipulated mouse strains listed in Table 2 also resulted in chimeric offspring with germ line transmission capability (Results not shown).

3. Germ Line Transmission After Diploid Aggregation

The germ line transmission capacity of 4 different mouse strains was tested after diploid aggregation with 8-celled embryos of the Swiss Webster strain (Table 4). All of the ES cell lines tested by diploid aggregation were able to produce chimeric offspring with germ line transmission capacity. overall, between 5-15% of all embryos reimplanted after diploid aggregation resulted in live offspring with an ES cell contribution. The percentage of chimerism of all offspring born with an ES cell contribution was very high. All chimeric mice born after diploid aggregation of ES cells from C57BL/6N #25, C57BL/6N #28, C57B1/6J-HPRT #2, 129/SvEv #4, 129/SvEv #11, 129/SvEv #17 with embryos of the Swiss Webster strain had 100% chimerism. After diploid aggregation with the 129SvEv #7 ES cell line, 3 out of 5 chimeric animals born, were 100% chimeric for the ES cell line. Fifty percent of all animals born after diploid aggregation with CBA/CaOla #4 ES cells showed a 100% chimerism.

4. Germ Line Transmission After Tetraploid Aggregation

Several of the established ES cell lines were tested for their germ line transmission capability after tetraploid aggregation (cfr. Table 5).

Embryos for the tetraploid component of the chimeras were obtained from the ROSA26 mice, which expresses LacZ ubiquitously and throughout the entire development and adulthood.

Four of the established 129SvEv ES cell lines, tested in tetraploid aggregation produced completely ES cell derived offspring after tetraploid aggregation. Between 3 and 30% of the reimplanted embryos produced live offspring. Tetraploid aggregation of ES cell line #7 of the 129SvEv strain at passage 17 was carried out with ROSA 26 tetraploid blastomeres and 13 and 10 aggregates were transferred to two foster mothers, yielding 3 and 4 live offspring respectively. All 7 offspring were totally ES cell derived and fertile, having produced 1 to 4 litters comprising of 11 to 40 pups.

Seven pups (12% of all reimplanted embryos) were born after tetraploid aggregation of a selected C57BL/6 ES cell line at passage 12 with ROSA 26 tetraploid blastomeres. Two males, randomly selected out of the 7, showed germ line transmission.

Improved ES cell medium and derivation conditions for murine ES allowed to derive ES cells with germ line transmission capability after tetraploid aggregation from CBA/CaOla mice, a strain previously believed to be non-permissive to ES cell derivation.

With the availability of these ES cells, it is possible to induce mutations in the genetic background of choice and to analyze the induced mutation without time-consuming inbreeding. Furthermore, these ES cells can be used to generate transgenic ‘gain-of-function’ mice since it is both inefficient and expensive to produce transgenic mice via pronuclear microinjection in backgrounds other than FVB and C57BL/16.

### Example 4

Larger Scale Production and Evaluation of Improved ES Cell Medium

1. Larger Scale Production of Rab9# 19 Conditioned Medium

The cryopreserved Rab9 #19 cells (10⁷ cells) were thawed and seeded in 2 T175 flasks. Upon confluence, the cells were passaged in a 1200 cm² cell factory at a density of 25 000 cells/cm². Upon confluence, the cells were harvested and seeded in a 3L bioreactor containing 1L of Improved ES cell medium and 2.47 g of cytodex 3 at a density of 15 000 cells/cm².

The bioreactor (Applicon, 3L) was equipped with a marine type impeller and a perfusion system. Aeration was performed through a microsparger. The pH was continuously monitored and maintained at 7.4 by addition of 1N NaOH.

The suspension was sampled daily to monitor the cell growth and LIF concentration. When the LIF concentration reached values between 15 and 20 ng/ml (approximately at day 3-4), the perfusion was initiated at a rate of about 0.5 L/day. The culture was maintained for 30 days. The perfusion rate was adapted over the life of the culture to result in a LIF concentration of 18-20 ng/ml. The perfusate was collected at 4° C. by 3 days pool.

According to an improved embodiment of the invention Improved ES cell medium can subsequently be constituted by adding to each liter collected (3 day pool) perfusate, 80 ml foetal bovine serum, 17 ml non-essential amino acids, 5μl β mercaptoethanol, 1.25 ml insulin, 80 to 130 ml basic ES cell medium (to adjust the LIF to a final concentration of 14 to 15 ng/ml). The Improved ES cell medium of the invention is preferably filtered on 0.22 micron cellulose acetate filters and frozen at −80° C. Upon usage 20 ml glutamine is added per liter Improved ES cell medium.

2. Derivation of Mouse ES Cell Lines from Two Inbred Mouse Strains with Improved ES Cell Medium Produced on a Larger Scale

The quality of this Improved ES Cell Medium (produced on a larger scale) was tested by evaluating it's potential to allow the establishment of ES cell lines from C57BL/GNTacfBr (Taconic, Germantown, N.Y., USA) and FVB/NTacfBR (Taconic) mouse.

When 3.5 days old blastocysts were collected from C57BL/6N and FVB/N mouse and ES cells were derived according to the procedures described earlier, respectively 58% and 50% of the blastocysts gave rise to an ES cell line.

TABLE 1
Establishment of ES cell lines from 11 inbred
and 1 outbred (Swiss Webster) mouse strains.

| | | | Established | |
|---|---|---|---|---|
| | Blastocysts | | ES cell | Efficiency |
| Mouse strain | cultured | Date | lines* | in % |
| 129/Sv Ev | 18 | May 1997 | 11 | 61 |
| 129/SvJ | 12 | May 1998 | 7 | 58 |
| C57BL/6N | 30 | May 1997 | 12 | 40 |
| C57BL/6JOla | 12 | August 2000 | 7 | 58 |
| C57BL/6J- | 25 | January 1998 | 8 | 32 |
| HPRT | | | | |
| CBA/CaOla | 12 | December 1997 | 8 | 66 |
| DBA/2N | 16 | June 1998 | 6 | 37 |
| DBA/1Ola | 36 | December 1998 | 4 | 11 |
| C3H/HeN | 48 | October 2000 | 15 | 31 |
| BALB/c | 37 | July 1998 | 2 | 5 |
| FVB/N | 18 | May 1998 | 4 | 22 |
| Swiss | 85 | December 1998 | 6 | 7 |
| Webster | | | | |

*10 or more passages.

TABLE 2
Establishment of ES cell lines from genetically
manipulated mouse strains.

| | | | Blastocysts | Established ES | Efficiency |
|---|---|---|---|---|---|
| Mouse strain | Gene inactivated | Date | cultured | cell lines* | in % |
| 129/Sv × C57B16 | Nitric oxide | December 1999 | 12 | 6 | 50 |
| | synthase | | | | |
| 129SvJ × 129Sv Pas | Vit D receptor | August 1998 | 10 | 6 | 60 |
| C57BL/6N | ApoE | August 1998 | 18 | 2 | 11 |
| 129SvJ × 129SvPas | Survivin | January 2000 | 100 | 44 | 44 |
| Swiss Webster × | HIF1α | March 1998 | 57 | 20 | 35 |
| (129SvJ × 129SvPas) | | | | | |
| 87.5% C57BL/6 × | Tissue Factor | September 1997 | 45 | 26 | 58 |
| (12.5% 129SvJ × 129SvPas) | | | | | |

*10 or more passages.

TABLE 3
Production of chimeric mice after blastocyst injection with established ES cells.

| | ES cell | | Passage | Blastocysts | Animals | | Germ line |
|---|---|---|---|---|---|---|---|
| Strain | line | Date* | # | injected | born | Chimeras | Transmission** |
| 129SvJ | #3 | September 1999 | 10 | 20 | 4 | 2 | 1/1 |
| | | September 1999 | 14 | 33 | 11 | 8 | nd |
| 129SvJ | #4 | September 1999 | 11 | 33 | 6 | 2 | 1/1 |
| | | October 1999 | 15 | 32 | 3 | 1 | 1/1 |
| 129SvJ | #7 | September 1999 | 16 | 30 | 7 | 6 | 2/3 |
| C57Bl/6J-HPRT | #2 | July 1999 | 12 | 60 | 40 | 23 | 1/3 |
| DBA/2N | #8 | July 1999 | 15 | 60 | 17 | 4 | 1/1 |
| DBA/1 Ola | #36 | October 1999 | 11 | 36 | 10 | 0 | 0/0 |
| | | December 1999 | 21 | 43 | 14 | 9 | 1/2 |
| BALB/c | #17 | October 1999 | 17 | 36 | 16 | 11 | 3/3 |
| | #29 | October 1999 | 17 | 39 | 12 | 3 | 1/1 |
| | | October 1999 | 18 | 49 | 18 | 13 | 1/1 |
| FVB/N | #17 | October 1999 | 10 | 50 | 18 | 3 | 1/1 |
| | | December 1999 | 16 | 34 | 12 | 5 | 2/2 |
| Swiss Webster | #43 | November 1999 | 12 | 36 | 9 | 5 | 1/2 |
| | | August 1999 | 13 | 37 | 8 | 3 | 1/1 |
| | | November 1999 | 14 | 47 | 3 | 1 | nd |
| | | December 1999 | 15 | 33 | 9 | 2 | 1/1 |
| Swiss Webster | #44 | July 1999 | 14 | 15 | 7 | 4 | 1/1 |

*Date of first litter born
**Successes versus total tested

TABLE 4
Production of chimeric mice with germ line transmission capability
after diploid aggregation with the established ES cell lines.

| | ES | | | | | Chimaeras | |
|---|---|---|---|---|---|---|---|
| | cell | | Passage | # embryos | Animals | (% | Germ line |
| Strain | line | Date* | no. | reimplanted | born | chimerism) | transmission** |
| 129SvEv | #4 | June 1997 | 12 | 59 | 24 | 3 M (100%) | 3/3 |
| | | | | | | 1 F (100%) | |
| 129SvEv | #7 | June 1997 | 13 | 32 | 12 | 3 M (100%) | 3/4 |
| | | | | | | 1 M (60%) | |
| | | | | | | 1 F (10%) | |
| 129SvEv | #11 | February 1998 | 12 | 98 | 16 | 8 M (100%) | 1/1 |
| 129SvEv | #17 | February 1998 | 12 + 13 | 82 | 10 | 5 M (100%) | 1/1 |
| C57BL/6N | #25 | February 1998 | 13 | 99 | 47 | 19 M (100%) | 1/1 |
| C57BL/GN | #28 | February 1998 | 13 | 89 | 40 | 8 M (100%) | 1/1 |
| C57BL/6J-HPRT | #2 | February 1998 | 13 | 65 | 26 | 5 M (100%) | 1/3 |
| | | April 1998 | 14 | 114 | 28 | 6 M (100%) | 1/2 |
| CBA/CaOla | #4 | February 1998 | 12 | 80 | 23 | 4 M (100%) | 2/3 |
| | | | | | | 2 M (50%) | |
| | | | | | | 1 F (50%) | |
| | | | | | | 1 F (40%) | |

*Date of first litter born
**Successes versus total tested

TABLE 5
Production of chimeric mice with germ line transmission capability after
tetraploid aggregation with the established ES cell lines.

| | ES cell | | Passage | # embryos | | Germ line |
|---|---|---|---|---|---|---|
| Strain | line | Date* | no. | reimplanted | Animals born | transmission** |
| 129SvEv | #4 | October 1997 | 13 | 10 | 1 (10%) | 0/1 |
| 129SvEv | #7 | April 1998 | 12 | 53 | 11 (21%) | 2/2 |
| | | April 1998 | 16 | 66 | 10 (15%) | 2/2 |
| | | September 1997 | 17 | 23 | 7 (30%) | 7/7 |
| 129SvEV | #11 | March 1998 | 12 | 132 | 7 (5%) | 1/1 |
| 129SvEv | #17 | March 1998 | 12 | 139 | 5 (3%) | 1/1 |
| C57BL/6N | #25 | January 1998 | 12 | 56 | 7 (12%) | 2/2 |
| CBA/CaOla | #4 | October 1997 | 11 | 67 | 1 (1.5%) | 1/1 |

*Date of first litter born
**Successes versus total tested

TABLE 6
Establishment of ES cell lines from two inbred
mouse strains with large scale produced Improved ES cell
medium.

| | Blastocytes | Established ES | Efficiency |
|---|---|---|---|
| Mouse strain | cultured | cell lines* | in % |
| C57BL/6N | 12 | 7 | 58 |
| FVB/N | 24 | 12 | 50 |

*10 or more passages.

1. Composition for maintenance and growth of a pluripotent and germ line-competent mammalian embryonic stem (ES) cell line, which composition consists of a basal cell medium, which comprises high glucose DMEM, non-essential amino acids, glutamine, β-mercaptoethanol, insulin and fetal bovine serum or equivalents thereof, which basal cell medium is conditioned by a fibroblast cell clone that produces Leukemia Inhibitory Factor (LIF).

2. The composition according to claim 1, wherein the basal cell medium comprises the following compounds in amounts sufficient to maintain ES cells for prolonged periods in culture:
1) DMEM high glucose;
2) penicillin/streptomycin;
3) non essential amino acids;
4) glutamine;
5) β-mercaptoethanol; and
6) foetal bovine serum.

3. The composition of claim 1 or 2, wherein the LIF producing fibroblasts are immortalized rabbit fibroblasts.

4. The composition of claims 1-3, wherein the immortalized fibroblasts have been transfected, transformed or infected by a vector overexpressing a LIF gene.

5. The composition of claim 4, wherein the LIF gene is a rabbit LIF gene.

6. The composition of the claims 1-5, wherein the fibroblast cell line used for conditioning is the Rab9 #19 cell line, which has been deposited with the Belgian Coordinated Collection of Microorganisms, under accession number LMBP 5479 CB.

7. The composition of claims 1-7, comprising per each liter perfusate of the LIF producing cell line, added volumes of 50 to 120 ml, preferably 80 ml foetal bovine serum, 10 to 25 ml, preferably 17 ml non-essential amino acids, 2 to 8 μl, preferably 5 μl β-mercaptoethanol, 0.5 to 2.5 ml, preferably 1.25 ml insulin, 80 to 130 ml basal ES cell medium (to adjust the LIF to a final concentration of 14 to 15 ng/ml).

8. The composition of claim 7, wherein the basal ES cell medium consists of 400 to 600 ml, preferably 500 ml DMEM high glucose, 0 to 15 ml, preferably 13 ml penicillin/streptomycin, 10 to 15 ml, preferably 13 ml non essential amino acids, 10 to 15 ml, preferably 13 ml glutamine, 5 to 10 μl, preferably 6.3 μl β-mercaptoethanol, 50 to 100 ml, preferably 70 ml foetal bovine serum, neutral pH of preferably 7.4.

9. The composition as claimed in claims 1-8 for use in the production of pluripotent embryonic stem (ES) cell lines.

10. A process of culturing mammalian ES stem cells to obtain pluripotent and germ line-competent ES cells, wherein the culturing of the mammalian ES stem cells is at least partially performed in a composition as claimed in claims 1-8.

11. The process of claim 10, comprising the steps of:
a) culturing cells of blastocyst stage embryos;
b) culturing isolated inner mass cells; and
c) passaging the inner mass cells periodically in a composition as claimed in claims 1-8.

12. The process of claim 11, wherein the inner mass cells are periodically passaged for at least 8 times.

13. The process according to any of the claims 10 to 12, further comprising the step of producing transgenic animals.

14. Embryonic stem (ES) cell line with germ line transmission capability.

15. The cell line according to claim 10, which has germ line transmission capability after 11 or more passages.

16. The cell line of claim 14 or 15, obtainable by the process of any of the claims of 10 to 12.

17. The cell line according to claims 14-16, wherein the cell line is a murine cell line.

18. The cell lines according to claim 17, wherein the cell line has been derived from cells or tissues with 129/SvEv, C57BL/6N, C57BL/6J-HPRT, BALB/c, CBA/CaOla, 129/SvJ, DBA/2n, DBA/1 Ola, C3H/HeN, C57B1 6JOla, FVB or Swiss Webster genetic backgrounds.

19. The cell line of claim 18, which has a germ line transmission capability after 11 or more passages.

20. The cell line as claimed in claims 14-19, wherein the cell line is cultured in a composition as claimed in claims 1-8 supplemented with cytokines and growth factors.

21. Embryonic stem (ES) cell line as claimed in any one of the claims 14-20, characterized by three dimensional colony formation, positive staining for alkaline phosphatase and negative staining for cytokeratin 18 and vimentin after more than 10 passages.

22. Embryonic stem (ES) cell line as claimed in any one of the claims 14-21 for use in the generation of chimeric or ES cell derived animals.

23. Embryonic stem (ES) cell line as claimed in any one of the claims 14-21 for use in the gene alteration by homologous or non-homologous recombination.

24. Embryonic stem (ES) cell lines as claimed in any one of the claims 14-21 for use in the generation of animals with gene alteration via germ line transmission.

25. Use of ES cell lines according to any of the claims 14-21 for the generation of chimeric animals.

26. Use as claimed in claim 25 for the generation of chimeric animals following blastocyst injection into recipient blastocysts or embryo aggregation or nuclear transfer.

27. Use or differentiation of cell lines according to any of the claims 14-21 for the study or isolation of (novel) genes.

28. Use of ES cells according to any of the claims 14-21 for the expression or overexpression of genes.


SEQUENCE LISTING (1 sequence)

SEQ ID NO: 1 - DNA, 594 bp, Artificial Sequence
  CDS (1)..(591): Phage plaques of Sau 3A partial rabbit genomic library encoding the rabbit LIF protein.
ggagtcgtgcccctgctgctggtcttgcactggaaacccggggcggggagctgaccccttcccatcaaccccgtcaacgccacctgcaacacacaccacccatgccccagcaacctcatgagccagatcaggagccagctggcacagctcaatggcactgccaacgccctctttattctctattacacagcccaaggggagccgttccccaacaacctggacaagctgtgcggccccaatgtgacggacttcccgcccttccacgccaacggcacggagaaggtcaggctggtggagctgtaccgcatcgtcgcctaccttggcaccgccctgggcaacatcacccgggaccagaagaccctcaaccccacggcgcacagcctgcacagcaaactcaacgccacggcggacacgctgcggggcctgcttagcaacgtgctgtgccgcctgtgcagcaagtaccacgtggcccacgtggacgtggcctatggcccggacacctcgggcaaggacgtcttccagaagaagaagctggggtgtcagctgctgggaaaatacaagcaggtcatggccgtgttggcgcaggccttctag